(19) United States
(12) Patent Application Publication
(10) Pub. No.: US 2017/0219450 A1
(43) Pub. Date: Aug. 3, 2017

(54) PRESSURE/FORCE SENSORS FOR MEASURING FLUID PRESSURES; CALIBRATION METHODS FOR FLUID PRESSURE/FORCE SENSORS; FLUID DRAINAGE SYSTEMS

(71) Applicant: Aqueduct Critical Care, Inc., Bothell, WA (US)

(72) Inventors: Marek SWOBODA, Philadelphia, PA (US); Barry Lutz, Seattle, WA (US)

(21) Appl. No.: 15/487,831

(22) Filed: Apr. 14, 2017

Related U.S. Application Data
(63) Continuation-in-part of application No. 15/029,592, filed on Apr. 14, 2016.
(60) Provisional application No. 61/961,403, filed on Oct. 15, 2013.
(60) Provisional application No. 62/322,725, filed on Apr. 14, 2016.

(51) Int. Cl. G01L 27/00; A61B 5/03; A61B 5/08; A61B 5/0215

(52) U.S. Cl. CPC G01L 27/002; A61B 5/02156; A61B 5/031; A61B 5/08; A61B 2562/0247

(57) ABSTRACT

In one aspect, the disclosure provides a pressure sensor that wirelessly provides force/pressure data to a wireless receiver. The pressure sensor includes a first fluid-responsive membrane configured to be exposed to a region, such as a body fluid, whose pressure is being monitored. A force transducer for measuring this pressure is movable toward and away from the flexible membrane and may be oscillated, either out-of-contact with the first fluid-responsive membrane or in-contact therewith, for static/dynamic pressure sensor calibration. An actuator for displacing/oscillating the force transducer is located within the internal housing. Specific pressure transducers, fluid drainage systems, implantable devices and (at least partially) external sensing devices are disclosed. Calibration techniques, including recalibration to adjust for device drift and to clear biofouling are disclosed.

### REFERENCE TO RELATED PATENT APPLICATIONS

This application is a continuation-in-part of U.S. patent application Ser. No. 15/029,592, filed Apr. 14, 2016, which is a U.S. national phase patent application of PCT/US2014/060401 filed Oct. 14, 2014, which claims the benefit of U.S. Patent Application No. 61/961,403, filed Oct. 15, 2013. This application also claims the benefit of U.S. Patent Application No. 62/322,725, filed Apr. 14, 2016. The disclosures of these priority applications are incorporated herein by reference in their entireties.

### BACKGROUND

1. Field

In one aspect, this disclosure generally relates to medical devices for monitoring pressure present in fluid of a living being, e.g., intracranial pressure, of a living being. In another aspect, this disclosure generally relates to medical devices for regulating body fluid in a living being, e.g., body fluid drainage systems, which may be implemented internally and/or externally of the living being.

2. Description of Related Art

Implantable sensors are important diagnostic devices which help measure physiological parameters that are difficult or even impossible to measure noninvasively. However, implantable devices pose several problems for the designer. They have to be biocompatible so they do not harm the patient over a long or short term, and they cannot trigger physiological or patho-physiological reactions (e.g., immunological reactions) which can compromise their ability to perform measurements.

Another set of problems stems from engineering requirements. The stability requirements for the implantable sensor are more strict than those for the noninvasive devices since they cannot be calibrated at will, or at least, the calibration process is usually more challenging compared to other devices. Calibration of externally placed sensors, however, is also challenging.

Long term implantable (and externally placed) pressure sensors carry inherent problems producing sensor drift and error and affecting their stability. First, short term temperature fluctuations change the internal temperature, thus changing the internal pressure. These pressure changes affect the pressure differential between the internal pressure of the device and the external pressure (e.g., body fluid pressure, intracranial pressure, ICP). Another short-term factor affecting sensor accuracy may include the change in the amount of gas inside the sensor body (e.g., gas absorption due to oxidation or gas release from materials inside the capsule). These types of changes can also add to or subtract from forces acting on the pressure/force transducer by changing forces acting on the membrane separating the inside of the sensor from the external environment. Another source of drift might be related to sensor aging. However, the use of solid-state components improves the longevity and performance of the sensors. In addition, natural body responses may cause protein deposits on the surface of the membrane, thereby changing the effective stiffness of the membrane detecting pressure. This change in effective stiffness may change the sensitivity of the device or even entirely block the detection of external pressure. This type of problem is usually associated with long-term changes.

A typical solution to these problems is to utilize two identical sensors which respond to temperature and aging the same way. One sensor is usually exposed to the measured quantity while the reference sensor is only exposed to conditions inside the sensor housing. The resulting signal is calculated as a difference between the reference signal and the second sensor. However, this solution has several drawbacks: e.g., the reference pressure in the reference transducer has to be kept constant.

The above-listed problems (assuming that the membrane by itself does not generate any stress on the sensor regardless of the displacement, i.e., an ideal membrane) can cause the output-input characteristic of the sensor to shift up or down (see FIG. 1A), or to rotate about certain point(s), changing the slope of the characteristic (FIG. 1B). In particular, plot 1 of FIG. 1A depicts the undisturbed input-output characteristic. Plot 2 of FIG. 1A depicts the input-output characteristic of the internal pressure (i.e., inside the sensor body), which is lowered. Plot 3 of FIG. 1A depicts the input-output characteristic if the internal pressure is elevated.

Every sensor carries an inherent risk of drifting with time. The active element of the sensor (e.g., piezoresistive element or die) changes its properties with time, temperature etc. FIG. 2 depicts the variance of output vs. measured quantity (e.g., pressure) as temperature changes. The lower line 2A in FIG. 2 represents the normal operation curve of the die when operating at a temperature T₁. The slope of this line 2A represents the sensitivity of the sensor at that temperature. If the temperature is increased, the piezoresistive die's response to changes in pressure also changes (see upper line 2B in FIG. 2); in particular, the sensitivity changes and also an offset component is introduced. Such factors can be resolved by hardware and, typically, sensor housings are constructed with built-in compensation. However, such solutions increase the size of the sensor and the power consumption.

A variety of medical conditions can cause the collection of excess body fluids within the human body. Drainage systems for regulating the drainage, and pressure, of excess body fluids are known in the art and are described, for example, in the following US and PCT International patent publications, which are incorporated herein by reference in their entireties: WO 2011/116393; US 2012/0302938; WO 2015/109260; and WO 2015/157320. FIG. 3A, herein, illustrates a schematic view of an internal body fluid drainage system implanted within a patient and used to measure pressure and to drain body fluid in accordance with these disclosures; FIG. 3B, herein, illustrates a schematic view of an external body fluid drainage system used to measure pressure and drain body fluid in accordance with these disclosures.

Measuring the pressure of internal fluids and regulating the drainage of internal fluids under abnormal conditions is challenging with both in situ and external sensors. One of the physiological parameters which is difficult to measure noninvasively is ICP. ICP can be an important parameter in monitoring hydrocephalic patients, or traumatic brain injury (TBI) victims, and it is also an important measurement in connection with drainage systems for regulating the drainage, and pressure, of excess body fluids in the body. Since cerebrospinal fluid (CSF) is enclosed in a semi-closed system (i.e., the skull), the forces exerted by it are counterbalanced by a rigid structure of bones and, to some extent, by a semi rigid structure of the spinal channel. In a mechanical sense, there is no direct link (except for some small vessels which are difficult to utilize due to their anatomical nature) between the cerebrospinal fluid and the external environment. Thus, both implantable sensors and external sensors communicating with the CSF outfitted with a reliable means of calibration would be a valuable addition to neurosurgical armamentarium.

U.S. Patent Publication No. 2013/0247644 (Swoboda, et al.) discloses a calibration system and method for an implantable pressure sensor having wireless communication capability. However, there remains a need for pressure sensors capable of providing a more accurate reading of the internal fluid pressures, and for calibration methods for such pressure sensors.

All references cited herein are incorporated herein by reference in their entireties.

### BRIEF SUMMARY

Pressure sensors for detecting a fluid pressure (e.g., intracranial pressure (ICP), blood pressure, lung pressure, or a non-body-related pressure, etc.) and for monitoring pressure present in fluid are disclosed. In some embodiments, pressure sensors are implantable; in some embodiments, pressure sensors may be located externally of a living being and may contact fluid present, e.g., in a catheter or tube. In some embodiments, a pressure sensor comprises: a sensor housing comprising one area having a first membrane responsive to fluid pressure and configured to contact the fluid pressure desired to be measured; and wherein the sensor housing further comprises sensor electronics including a movable force transducer configured to contact the first membrane for detecting a response (e.g., movement) of the first membrane when the membrane is exposed to the fluid pressure and changes in fluid pressure. In some embodiments, the force transducer is positioned to be moved into and out of contact with the first membrane for statically and/or dynamically calibrating the pressure sensor. In some embodiments, the first membrane has an external surface configured for placement in contact with a fluid whose pressure is desired to be measured, and the force transducer is positioned to contact an opposing surface of the first membrane (e.g., internally in the sensor housing) for detecting force exerted on the first membrane by the fluid.

In some embodiments, the sensor housing additionally comprises a reference membrane responsive to a reference fluid (e.g., a liquid or gas) having an external surface configured for contacting a reference fluid. In some embodiments, the sensor housing is sealed, while in some embodiments the sensor housing may be vented to the external environment. In embodiments in which the sensor housing is sealed, the housing may be filled with a fluid (e.g., a liquid or a gas).

In some embodiments, the movable force transducer may be mounted to or otherwise associated with a fluid-responsive membrane. In some embodiments, the movable force transducer may have a known mass mounted to or otherwise associated with it. In some embodiments, the movable force transducer may be mounted on an actuator, such as a piezo-actuated actuator. In some embodiments, the movable force transducer is a piezo-electric cantilever actuator capable of repositioning the sensor/mass away from and out of contact with the first flexible membrane for drift recalibration or re-zeroing the sensor. In some embodiments, the actuator vibrates (or oscillates) the sensor/mass (alone, i.e., out of contact with the membrane), or the actuator vibrates the combination of the sensor/mass and the membrane (i.e., the sensor/mass in contact with the membrane), creating known forces that can be used to recalibrate the sensor. In yet additional embodiments, the actuator vibrates (or oscillates) the mass and generates known forces that can be used to recalibrate for drift and re-zero the sensor in one position and that generates known forces that can be used to recalibrate the sensor for biofouling in another position. In the disclosure herein, the terms vibrate and oscillate are used interchangeably.

Devices and methods for calibrating a pressure sensor used for detecting a pressure (e.g., intracranial pressure (ICP), blood pressure, lung pressure, etc.) present in fluid, such as fluid of a living being, are disclosed. The methods disclosed herein may additionally and alternatively be used for calibrating a pressure sensor used for detecting pressures present in fluids extraneous of and unrelated to fluids present in a living being. In some embodiments, a calibration method comprises: disposing a pressure sensor comprising a first fluid-responsive membrane forming a portion of the pressure sensor in contact with the fluid whose pressure is desired to be detected, measured or monitored; and displacing a force transducer mounted in a housing of the pressure sensor from a position in contact with the membrane to a position out of contact with the first fluid-responsive membrane, followed by re-calibration and/or re-zeroing of the sensor (at a zero-force point).

Additional calibration methods involve oscillating a force transducer (e.g., a force sensor, a mass having a known weight, and an actuator) while the force transducer remains in contact with, or out of contact with, the first fluid-responsive membrane at one or more frequency(ies) and amplitude(s), e.g., in a sinusoidal oscillation, while collecting force data. Oscillation of the force transducer may be accomplished, in some embodiments, by applying an oscillating electrical signal (e.g., voltage or current) to the actuator to cause the actuator, force sensor and mass to oscillate. Using this technique, the oscillation frequency is known, the physical amplitude of the movement of the sensor/mass may be predicted or detected (e.g., as described below), and a pressure sensor force is measured by the force sensor. The theoretical (calculated) pressure sensor force can be calculated as the magnitude F=ma, where m is the mass and a is the acceleration. For a sinusoidal motion with amplitude A and frequency ω, the physical motion (displacement) is given by x=A sin(wt), the velocity is v=Aw cos(wt), and the acceleration is a=−Aŵ2 sin(wt). So the magnitude (largest value) of each is |x|=A, |v|=Aw, |a|=Aŵ2, and |F|=mAŵ2. The measured force may thus be compared and related to the theoretical or calculated pressure sensor force to establish dynamic calibration characteristics.

In one embodiment, the force transducer may be positioned out of contact with the fluid-responsive membrane and oscillated while remaining out of contact with the first fluid-responsive membrane, which is in contact with the fluid. A first measured force data set may be collected while the force transducer is oscillated at a first frequency and a first amplitude, and a second measured force data set may be collected while the force transducer is oscillated at a different frequency and/or amplitude, e.g., a second frequency and a second amplitude. The theoretical pressure sensor force at the different frequency and amplitude oscillations may be determined (as described above) by measuring or predicting the amplitudes, and a multi-point calibration characteristic based on comparison of the measured and calculated forces of multiple force data sets may then be derived. Calibration of the force transducer to the correct for differences between measured and calculated forces provides more accurate pressure sensing. Additional force transducer oscillations at additional amplitudes and frequencies may be performed to derive additional dynamic calibration points and/or to establish calibration characteristics based on additional sets of measured and theoretical force data.

Pressure sensor error may be introduced as a result of fouling of the fluid-responsive membrane, e.g., by deposit of biomaterial or other types of contaminants on the membrane, which cause the membrane to inaccurately reflect the pressure of the liquid contacting the membrane. In one calibration method, the force transducer may be oscillated while remaining in contact with the first fluid-responsive membrane. A first measured force data set may be collected while the force transducer is oscillated at a first frequency and first amplitude, and a second measured force data set may be collected while the force transducer is oscillated at a different frequency and/or amplitude. The calculated pressure sensor force may be determined (as described above) by measuring or predicting the oscillation amplitudes, and a multi-point calibration characteristic based on the first and second data sets may then be derived. This calibration technique is particularly suitable for providing calibration of the sensor to detect biofouling and the extent of biofouling. Additional force transducer oscillations at additional amplitudes and frequencies may be performed to derive additional dynamic calibration points and/or to establish calibration characteristics based on multiple sets of measured and theoretical force data.

In yet additional oscillation frequency-based methods, calibration characteristics may be derived based on oscillation of the force transducer at mechanical or resonance frequencies. In other methods, calibration characteristics may be derived based on the motion resulting from quickly moving the sensor between two discrete positions by applying an actuation signal, such that the final position is out of contact with the sensing membrane, and the sensor oscillates without further actuation due to its own inertia and the mechanical resonance of the system.

Additional methods involve disposing a pressure sensor comprising a first fluid-responsive membrane forming a portion of an outer surface of the pressure sensor in contact with pressure present in fluid; positioning a force transducer on a second membrane spaced from the first membrane and forming an upper surface of an internal housing that is suspended by a third membrane to an inner wall of the pressure sensor and wherein the force transducer is in contact with the first membrane; displacing the force transducer to a first position to be out of contact with the first membrane and measuring the force required to achieve such a first position to form an initial calibration point; oscillating the force transducer at the first location and collecting a first set of displacement data as the force transducer is oscillated; deriving a first dynamic force calibration point from the first set of displacement data; displacing the force transducer to a second position that is even further out of contact with the first membrane than the first position and measuring the force required to achieve the second position; oscillating the force transducer at the second location and collecting a second set of displacement data as the force transducer is oscillated; deriving a second dynamic force calibration point from the second set of displacement data; and establishing a force calibration characteristic based on the first and second sets of displacement data.

In some embodiments, methods for calibrating a pressure sensor in situ within a living being for detecting a pressure (e.g., intracranial pressure (ICP), blood pressure, lung pressure, etc.) present at a location within the living being are disclosed. In some embodiments, methods for calibrating a pressure sensor located externally of a living being and in contact with pressure present in fluid are disclosed. In some embodiments, pressure sensors and calibration methods as disclosed herein are used in body fluid drainage systems in which the pressure sensor may be implanted within a body or positioned outside of a body, in contact with fluid whose pressure and/or flow is desired to be monitored.

### BRIEF DESCRIPTION OF THE DRAWINGS

The disclosures here are described in conjunction with the following drawings in which like reference numerals designate like elements and wherein:

FIG. 1A is a prior art graph that depicts how the input-output relationship changes with internal (i.e., inside the sensor body) pressure;

FIG. 1B is a prior art graph that shows how the input-output relationship changes due to protein buildup on the surface of the sensor;

FIG. 2 is a prior art graph showing how changes in temperature affect sensor sensitivity;

FIG. 3A shows a schematic view of a body fluid drainage system implanted within a patient in accordance with these disclosures;

FIG. 3B shows a schematic view of an external body fluid drainage system in accordance with these disclosures;

FIG. 4A is an enlarged cross-sectional view of a first embodiment of a sensor of the present disclosure showing the pressure/force transducer at the reference position;

FIG. 4B is similar to FIG. 4A but depicts the pressure/force transducer being displaced away from the upper membrane;

FIG. 5 is a timing diagram depicting the various activation commands to achieve calibration of a sensor of the present disclosure;

FIG. 6 depicts an exemplary sinusoidal vibration signal used for dynamic calibration;

FIG. 7 is a cross-sectional view of a second embodiment of a sensor of the present disclosure utilizing a “floating vibrator” for achieving dynamic calibration.

FIG. 8 is an enlarged cross-sectional view of the first embodiment of a sensor of the present disclosure including a transparent window for infrared communication;

FIG. 9 is an enlarged cross-sectional view of the second embodiment of a sensor of the present disclosure including a transparent window for infrared communication;

FIG. 10 is a block diagram showing the non-contact communication of a sensor with a remotely-located transceiver;

FIG. 11 depicts how a sensor can be positioned within the living being, e.g., within the head of a human, and how a sensor can communicate with an external hand-held portion;

FIG. 12 is a partial view of the head of a living being wherein an implantable sensor is placed within the subarachnoid space and which allows for infrared or radio communication with the handheld device;

FIG. 13 depicts one embodiment of an implantable sensor wherein the transducer-membrane assembly portion of the implantable sensor is placed at a distal end of a catheter and the transceiver portion of the sensor is positioned at a proximal end of the catheter for communicating with the hand-held device;

FIG. 14 is an enlarged view of the proximal end (A) and of the distal end (B) of the embodiment of FIG. 12;

FIG. 15 depicts an alternative position of an implantable pressure sensor being disposed just underneath the skin and against the skull;

FIG. 16 shows a schematic view of an ICP detection and monitoring device comprising a shunt valve for evacuating fluid (e.g., CSF), a catheter for CSF drainage, and a pressure sensor mounted to the catheter for monitoring pressure present the fluid;

FIG. 17 shows an enlarged schematic view of another embodiment of a pressure sensor (e.g., an ICP sensor) as disclosed herein that may be used in association with a shunt device, or a body fluid drainage device, or to detect and monitor fluid pressure, internally or externally of the location of fluid within a body;

FIG. 18A shows a schematic diagram illustrating a pressure sensor comprising an actuator/sensor/mass combination mounted and configured for oscillation in a sealed chamber having two flexible membranes, the actuator/sensor/mass combination being in a sensing position;

FIG. 18B shows a schematic diagram illustrating the forces acting on the sensing and reference membranes shown in FIG. 18A;

FIG. 18C shows a schematic diagram illustrating the pressure sensor of FIG. 18A in a static or dynamic calibration position;

FIG. 18D shows a schematic diagram illustrating a pressure sensor similar to that shown in FIG. 18A, comprising a combined actuator and sensor associated with a mass mounted for oscillation in a chamber in a static or dynamic sensing position;

FIG. 19A shows a schematic diagram illustrating a pressure sensor comprising an actuator/sensor/mass combination mounted for oscillation in a vented chamber; and

FIG. 19B shows a schematic diagram illustrating the forces acting on the sensing membrane shown in FIG. 19A.

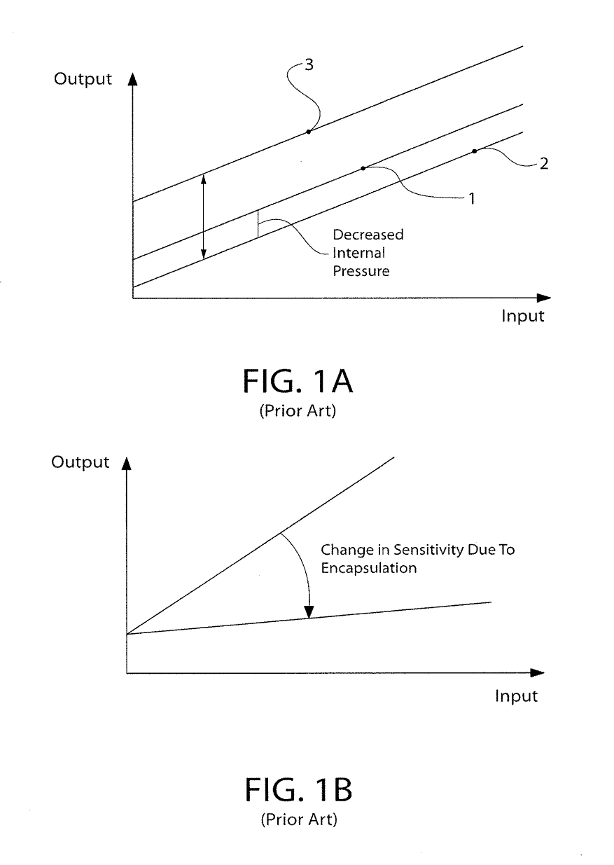

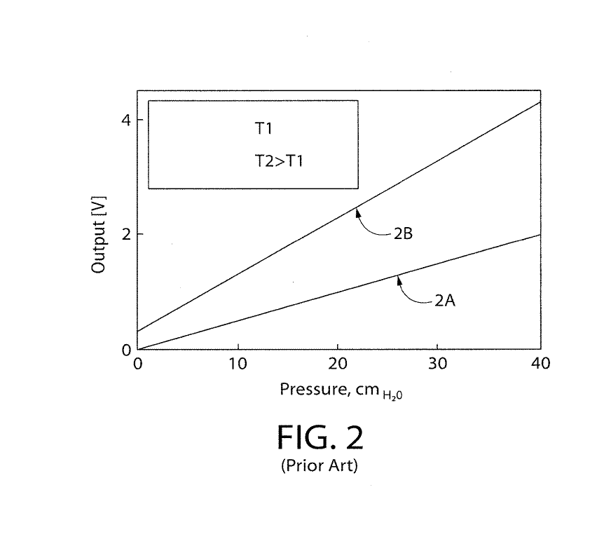

It will be understood that the appended drawings are not necessarily to scale, and that they present simplified, schematic views of many aspects of systems and components of the present invention. Specific design features, including dimensions, orientations, locations and configurations of various illustrated components may be modified, for example, for use in various intended applications and environments. It will also be appreciated that various features from one drawing may be omitted in alternative embodiments, and that various illustrated features may be used in combination with features from other drawings to provide additional embodiments, and that such combinations, whether resulting from omission or combination, are intended to be within the scope of the disclosure herein.

### DETAILED DESCRIPTION

The present disclosure, in some aspects, provides pressure sensors and calibration techniques that may be used within a body and/or externally of a body to detect and/or monitor the pressure and/or flow of fluid within a body or externally of a body. Any of the pressure sensors and calibration techniques described herein may be used with an implantable fluid detection, monitoring and/or drainage device, or with fluid detection, monitoring and/or drainage devices having both internally and externally positioned components, or with fully external systems. Any of the pressure sensors and calibration techniques described herein may also be used for fluid detection, monitoring and/or drainage in applications other than body fluid applications, such as industrial fluid measurement and detection applications, and the like.

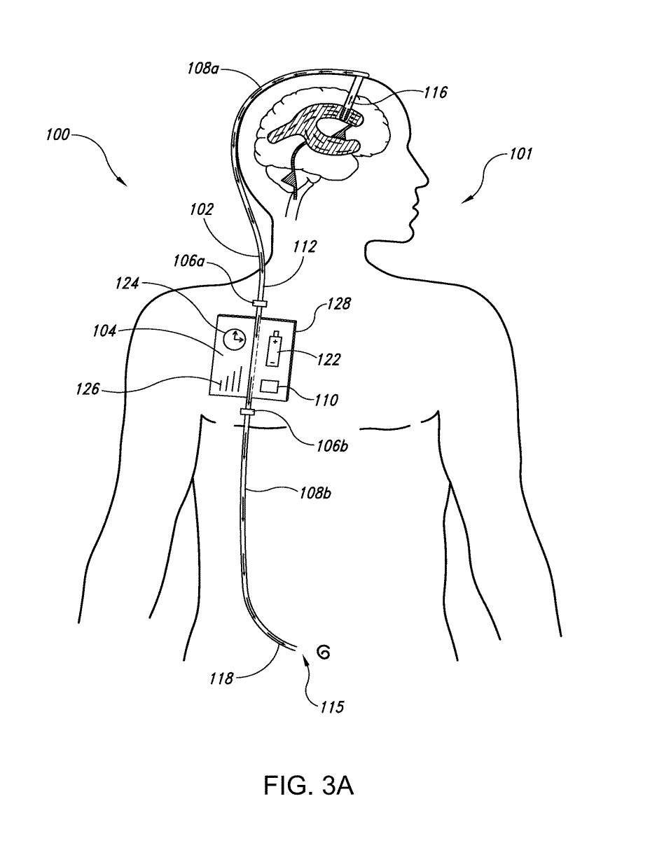

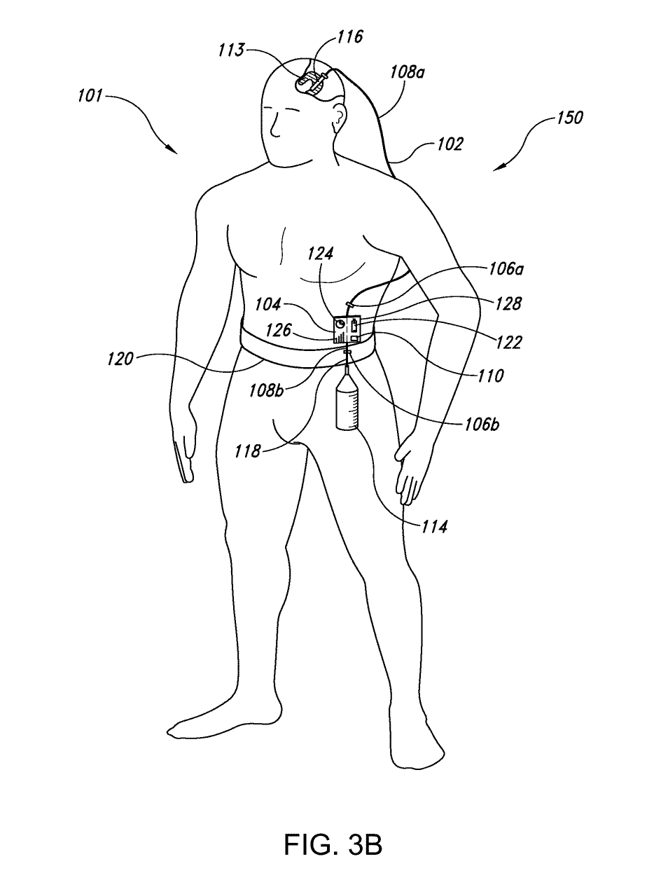

As shown in FIG. 3A, sensor assemblies 106 can be positioned proximate to the outlet and inlet of a valve device 104. In one embodiment, a first sensor assembly 106a can measure the flow rate and/or the pressure within the proximal port ion 108a before it enters the valve device 104 and the second sensor assembly 106b can measure the flow rate and/or pressure within a distal portion 108b as it exits the valve device 104. This information can be used to ensure the valve device 104 generates the desired drainage rate, to monitor patient orientation, to perform diagnostics on the drainage system, and/or derive other desired measurements or characteristics. In other embodiments, the drainage system 100 may include more or fewer sensors. For example, a pressure sensor may be positioned proximate inlet region 116 to measure ICP directly.

Sensor assemblies 106 may also be used to derive a pressure at a desired location spaced apart from the sensor assemblies 106. For example, sensor assemblies 106 positioned proximate the valve device 104 in the torso of a patient can be used to derive ICP. As shown in FIG. 3A, sensor assemblies 106 can be positioned on either side of the valve device 104 to measure pressure upstream and downstream of the valve device. The valve device may be positioned midway between the ventricle 113 and the outlet region 118, or in another region. In some embodiments, a pressure reference line can be coupled to the drainage system 100 and used to compensate for changes in patient position. A controller 110 is configured to read measurements taken from the sensors (e.g., pressure, flow rate, orientation, etc.), optionally store or communicate such measurements, optionally adjust the position of the valve device, and/or carry out algorithms to regulate fluid flow through the drainage system. Controller 110 may include logic to save power and/or to clear the valve device 104 of obstructions. The controller and a time keeping device 124 may be used to periodically flush the catheter 102 and/or to periodically run diagnostics routines. Controller 110 may be operatively coupled to a wireless communications link 126, allowing measurements, data, etc. from sensors to be monitored and/or analyzed remotely. Valve device 104, controller 110, and other devices or features may be enclosed within a housing 128 that may be implanted, e.g., subcutaneously in a patient, as schematically shown in FIG. 3A.

FIG. 3B schematically shows an external body fluid drainage system 150, similar to drainage system 100, having some elements implanted in a patient and some elements positioned externally of the patient. Drainage system 150 comprises catheter 102 having a proximal portion 108a and a distal portion 108b, with valve device 104 positioned between the proximal and distal portions, with sensor assemblies 106 and controller 110 operatively coupled to the sensors and valve. Like the internal drainage system 100, described above, external drainage system 150 can regulate fluid flow (e.g., CSF flow) using sophisticated and individualized methods, while operating as a low power system. An external receptacle 114 can be placed in fluid communication with outlet region 118 of catheter 102 for collection of excess body fluid. Housing 128 enclosing the valve device 104 and controller 110 may also be located externally, as shown. External components of the drainage system may alternatively be disposed on an external structure, such as an IV pole or another medical device or console.

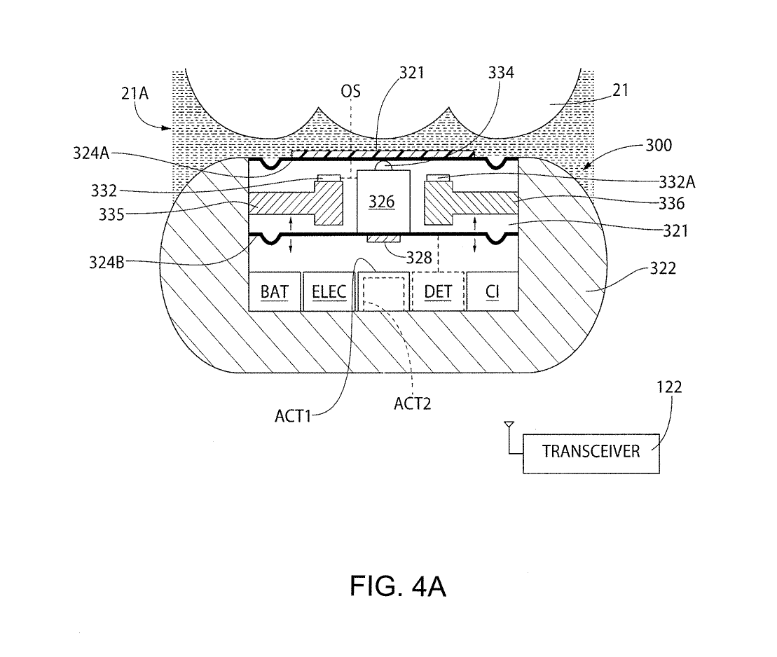

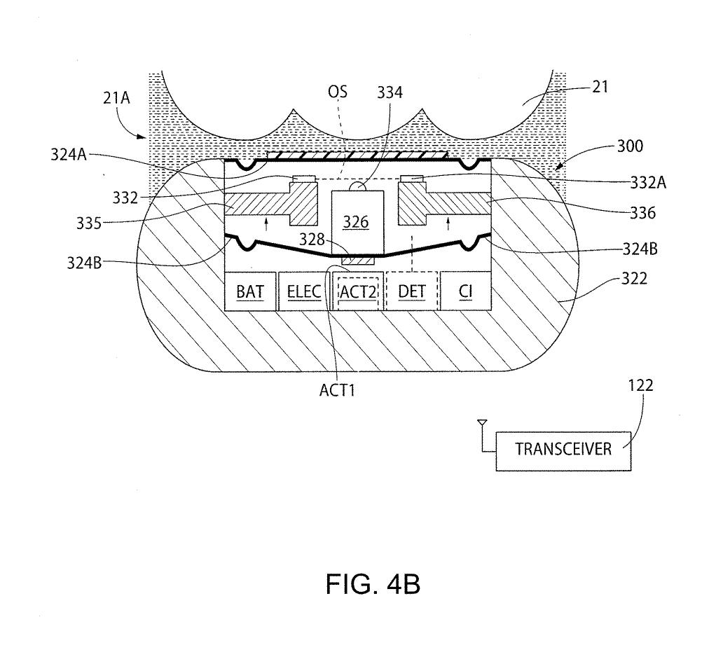

As shown in FIG. 4A, in some embodiments, the present disclosure involves a pressure sensor 300 and a remotely-located transceiver 122. The sensor 300 may be positioned within or adjacent or externally of the brain 21, such that the pressure sensor 300 is exposed to the cerebrospinal fluid (CSF) 21A. As a result, internal pressure CSF data obtained from the sensor 320 can be transmitted wirelessly to a remotely-located transceiver 122.

The pressure sensor 300 comprises a rigid housing 322 having a cavity 321 in which the internal components of the implantable pressure sensor 300 are located. The sensor 300 comprises at least one flexible membrane communicating with an external environment. In the embodiment illustrated, sensor 200 comprises a pair of elastic or flexible membranes 324A and 324B. The upper or outer flexible membrane or “CSF membrane” 324A forms a contact surface with the CSF 21A or another liquid pressure being monitored and experiences the intracranial pressure (ICP) or other liquid pressure; a rigid part of this flexible membrane is shown by element 321. The lower or internal flexible membrane 324B supports a pressure/force transducer 326 (e.g., a piezoresistive pressure sensor such as a low pressure sensor SM5103 or SM5106 by Silicon Microstructures Inc.). An actuator ACT (e.g., an electromagnet, microelectromechanical system (MEMS), piezoactuator, capacitive actuator, or other miniature actuator, etc.) can be used to drive the pressure/force transducer 326 up or down (i.e., to vibrate or oscillate the pressure/force transducer) within the cavity 323 for pressure sensor calibration purposes as well as to assess bio-fouling of the upper membrane 324A, and/or to dislodge build-up of bio material against the upper surface of the membrane 324A.

By way of example only, and as mentioned earlier, the miniature actuator ACT may comprise an electromagnetic actuator (e.g., micro-speaker L0008A-003 by Jong Gar Xin Tech. Co., Ltd.; micro device Spektrum SPMSA2005; etc.) that drives a paramagnetic, diagmagnetic or magnetic (e.g., ferromagnetic) plate 328. By way of example only, where a magnetic plate is used for the plate 328, the electromagnetic actuator may magnetically couple to a magnetic plate 328ss that is positioned on the lower side of the inner flexible membrane 324B. Furthermore, actuator ACT may even comprise two distinct actuators; one actuator ACT1 that provides a strong/slow force (also referred to as “large displacement”) on the magnetic plate 328 to displace the transducer 326 to engage or disengage from the upper flexible membrane 324A; and a second actuator ACT2 that provides a weak/fast force (also referred to as “small displacement”) that vibrates the transducer 326 at a desired frequency/amplitude, as will discussed later. Subsequent use of the term “actuator ACT” throughout this Specification is meant to encompass both scenarios: either a single miniature actuator that provide both types of forces or a pair of miniature actuators that provide the respective forces. Thus, by way of example only, FIG. 4A depicts the relative positions of the components when the actuator ACT is not energized, hereinafter known as the reference position or “x=0 displacement” position; FIG. 4B depicts the relative positions of the components when the actuator ACT has applied the strong/slow force to move the pressure/force transducer 326 away from the CSF membrane 324A.

It should be understood that the materials used in the sensor 300 (as well as the sensor 400, to be discussed below) may comprise bio-compatible materials when the sensors are used as implantable sensors, especially those sensor components/surfaces that are in direct contact with bio-material. As a result, the cross-sectional views shown in the figure do not necessarily indicate metal material and are used simply to indicate a cross-sectional of the component.

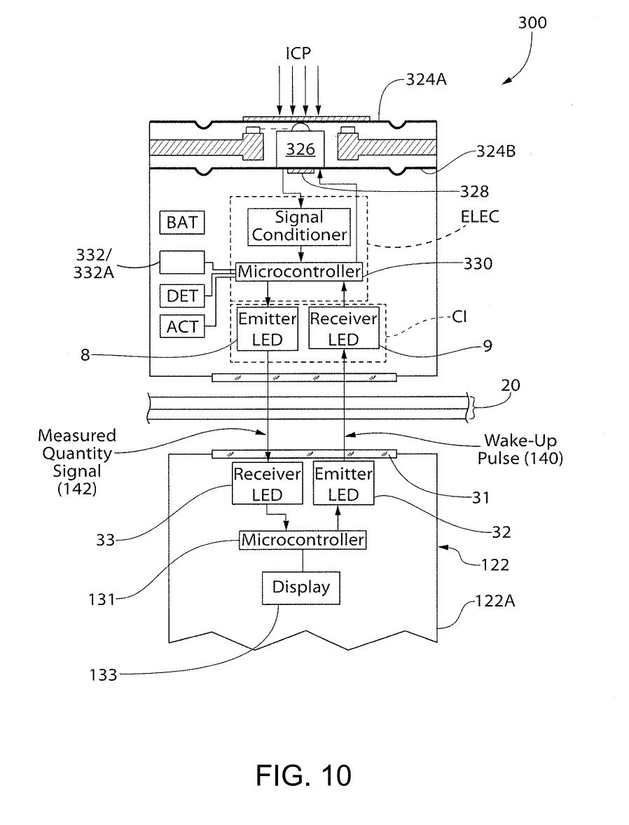

The sensor 300 may further comprise an internal power source (e.g., a battery BAT) for powering transducer electronics ELEC and a communication interface CI. The battery BAT may be a rechargeable type, receiving a recharge signal from a remotely-located transceiver 122. It should be understood that the battery BAT is by way of example only and that the sensor 300 may be a passive device that receives its electrical energy from a remotely-located transceiver 122 or another well-known external recharge device. The transducer electronics ELEC may further comprise a microcontroller 330 (FIG. 10; e.g., MSP430xG461x Mixed Signal Microcontroller by Texas Instruments). The microcontroller 330 controls/communicates with the actuator ACT, the communication interface CI, as well as with a sensor such as an optical sensor 332 or alternatively, a detector DET. When an optical sensor 332 is used, the sensor emits an optical signal OS whose interruption by a known mass 334, positioned on top of the transducer 326, alerts and indicates to the microcontroller 330 the displacement of the transducer 326; a target or reflector 332A is aligned with the emitter of the optical sensor 332 and both the emitter 332 and reflector 332A are fixed at aligned positions on respective support members 335 and 336. Alternatively, a detector DET (e.g., an infrared detector or other type of detector) can also be used to measure the overall displacement of the transducer 326. Cavity 321 may be sealed, and the media within the cavity 321 may comprise air, an inert gas, a vacuum, or a liquid. A signal conditioner takes the raw data signal from the pressure/force transducer 326 and conditions it (e.g., amplifies, filters, etc.) for use by the microcontroller 330.

### Sensor Calibration

The present disclosure solves some of the problems usually associated with pressure sensors designed and used for sensing body fluid pressures. It provides an easy calibration method which can reduce stability fluctuations and enable obtaining the correct measured value (e.g., ICP or another fluid pressure measurement), even if sensor offset or sensor sensitivity is altered. The sensor can be calibrated in situ once implanted or placed externally of a patient.

The present disclosure includes calibration techniques relating to the force transducer. In particular, one method involves calibrating the sensor in-place before the measured quantity (e.g., ICP or another pressure) reading is taken. Calibration techniques that are described with reference to implantable pressure sensors and in situ calibration may additionally be used in connection with pressure sensors used externally of a body, and with fluid drainage systems that may be implanted or located at least partially externally. Calibration techniques assure that the parameters that affect pressure readings are taken into account and therefore their effects are nullified.

In one embodiment for calibrating sensor 300, the sensor can be statically calibrated, or re-zeroed, by disengaging the transducer 326 from the upper membrane 324A. When the sensor is disengaged from the transducer, it is in a zero force condition and the sensor may be calibrated to zero force in this condition. In some embodiments, a force calibration characteristic may be derived using a “multi-point” calibration method where at least two verifiable force points are defined to create a calibration characteristic. In one embodiment, force points for defining a calibration characteristic can be measured using dynamic techniques by applying a vibratory force having a known frequency to the transducer and deriving the force from a plurality of displacement data, e.g. F=m·d²x/dt², where F is the force, m is the known mass and x is the displacement. In alternate embodiments, force points for defining a calibration characteristic can be measured dynamically by applying vibratory forces having varying amplitudes and/or frequencies to the transducer.

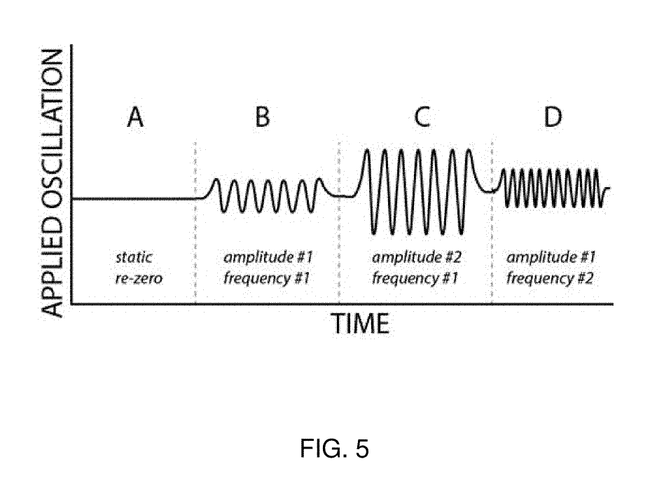

FIG. 5 shows a timing diagram depicting the various actuator ACT activation commands to achieve dynamic calibration of a sensor 300 and membrane assessment. As previously described, displacing the known mass 334 away from and out of contact with the upper membrane 324A before applying the oscillatory signal allows any tension in the upper membrane 324A caused by the engagement of the known mass 334 with the membrane 334A to be removed. Section A represents a static calibration or re-zeroing of the sensor, as described above, accomplished by moving the sensor out of contact with the upper membrane. By way of example only, if the miniature actuator ACT forms a single actuator, under section A, a positive DC current command may be applied to displace the mass 334/transducer 326 away from the upper membrane 324A, and a negative DC command can be applied to cause the pressure/force transducer 326 to move upward, causing the membrane 324A to move upward.

To perform a calibration protocol, a first oscillatory signal having a first known frequency and a first (known or detectible) amplitude can be applied to vibrate the transducer 326, as illustrated in Section B. Section C schematically shows application of a second oscillatory signal having the first frequency and a second (known or detectible) amplitude to vibrate the transducer 326. Section D schematically shows application of a third oscillatory signal having the second frequency and the first (known or detectible) amplitude to vibrate the transducer 326. Multiple force data sets are collected by measuring the force under different amplitude and frequency conditions, as described. In some embodiments, the force data sets acquired as described above are used to assess whether the membrane characteristics have changed significantly, as a result (for example) of build-up of contaminants such as bio-matter. Oscillation of the membrane at generally high frequencies may facilitate dislodging contaminants via the vibratory motion.

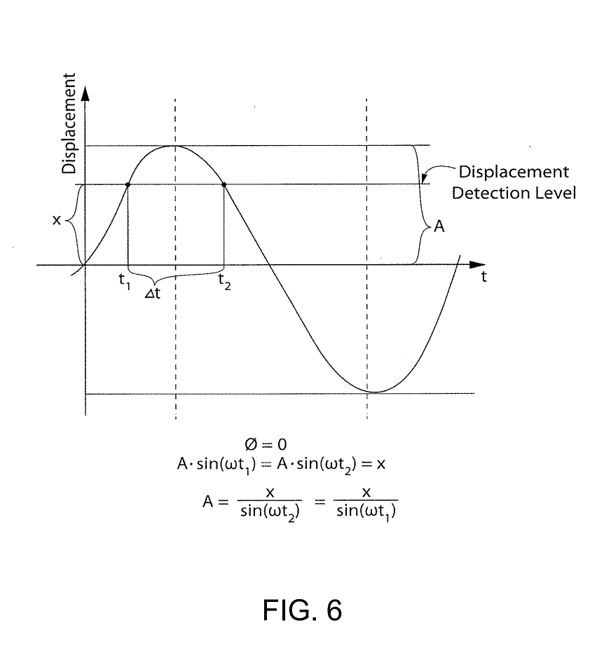

As shown in FIG. 6, the transducer 326 is vibrated with a known frequency in accordance with the sinusoidal characteristic A·sin(ω)t=x, where x is the vertical displacement. By taking the second derivative of this quantity, the force can be found. However, the only “knowns” are the frequency (ω), the phase (when the vibratory signal is initiated and terminated) and the displacement (the full displacement is the amplitude A), which can be measured at some point in the vibratory cycle via detection sensor 332/332A which is positioned at a predetermined “x” value away from a reference point. Detection sensor 332/332A may comprise an optical sensor that senses interruption by the mass; in another embodiment, the detection sensor may comprise a capacitive sensor that measures distance or displacement by means of a change in charge or current. Contact and/or magnetic sensors may also be used as detection sensors. In order to determine the second force point, it is necessary to determine the amplitude A of the vibratory motion of the mass, which as mentioned previously, may be unknown. One way to determine the amplitude A is to simply calculate it from a single measurement of t=t₁ when the top of the known mass 334 is detected, such as by an interruption of an optical signal OS; alternatively, and in addition, when the optical signal OS is restored as the top of the known mass 334 displaces downward, when t=t₂, the amplitude A can also be calculated. As mentioned previously, a plurality of similar amplitude calculations can be conducted over several cycles of the known frequency and from that data, the force calibration characteristic can be determined. In some cases, the amplitude A may be predicted based on a known response of the actuator/sensor/mass assembly to a known electrical input.

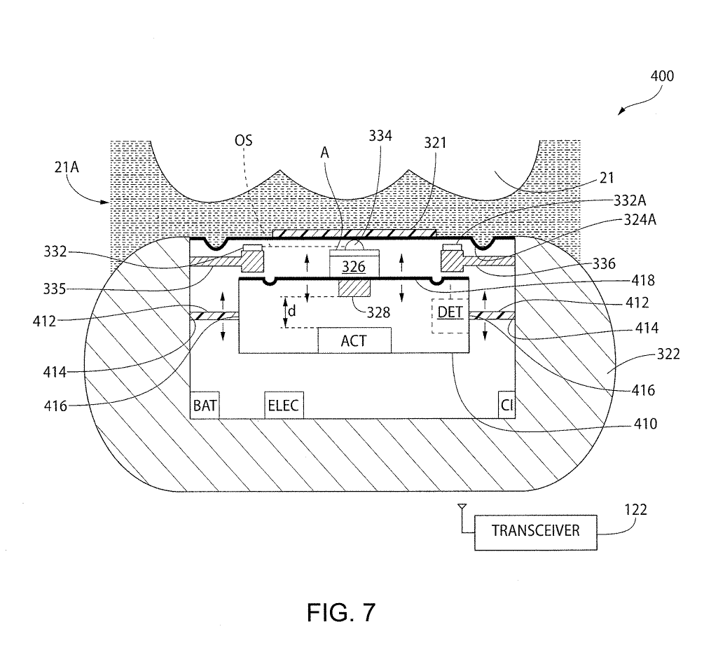

FIG. 7 depicts a second embodiment 400 of a pressure sensor, also referred to as a “floating vibrator.” In this configuration, the actuator ACT itself can be suspended on a third flexible membrane. In particular, the pressure sensor 400 comprises an internal housing 410 that is suspended within the cavity 321 by a third flexible membrane 412. This flexible membrane 412 is secured at its outer side 414 to the sensor housing 322 and to the internal housing 410 on its inner side 416. The upper surface of the internal housing 410 comprises a flexible membrane 418 to which the pressure/force transducer 326 is coupled to the membrane's 418 upper side and to which a plate 328 is coupled to the membrane's 418 lower side. As with the first embodiment 300, the pressure/force transducer 326 has a known mass 334 that is in contact with the upper or CSF membrane 324A when the actuator ACT is not energized. The optical sensor 332/332A and the detector DET operate similarly to that described with regard to the first embodiment 300, as well as the electronics ELEC and communication interface CI.

In operation, the actuator ACT moves plate 328 in a cyclical manner, as described earlier with regard to the first embodiment 300, using both DC and/or vibratory commands as also described earlier. The fluctuations in intensity of the oscillatory movement are cyclical and may be sinusoidal. However, unlike the first embodiment 300, because the actuator ACT is also suspended on the flexible membrane 412, when the actuator ACT moves the pressure/force transducer 326, the actuator ACT also displaces, thereby maintaining a constant separation distance “d” between the pressure/force transducer 326 and the actuator ACT. Acceleration caused by the up/down movement of the vibrator's membrane 418 generates calibrating forces acting on the sensing area SA of the pressure/force transducer 326 (due to the attached known mass 334). The membrane 412 on which the vibrator internal housing 410 is mounted can freely move due to initial forces, but can also be moved by additional actuators (not shown) in order to shift it upwards or downwards.

In some embodiments, calibration is performed internally in a sensing system, without requiring data transmission to remote devices or systems. In some embodiments, as mentioned previously, the sensors 300 or 400 may include a communication interface (CI) for wirelessly transmitting collected pressure data to the transceiver 122. As will be discussed in detail later, the communication format may include radio communication, infrared communication, etc., and the present disclosure is not limited to any particular communication methodology.

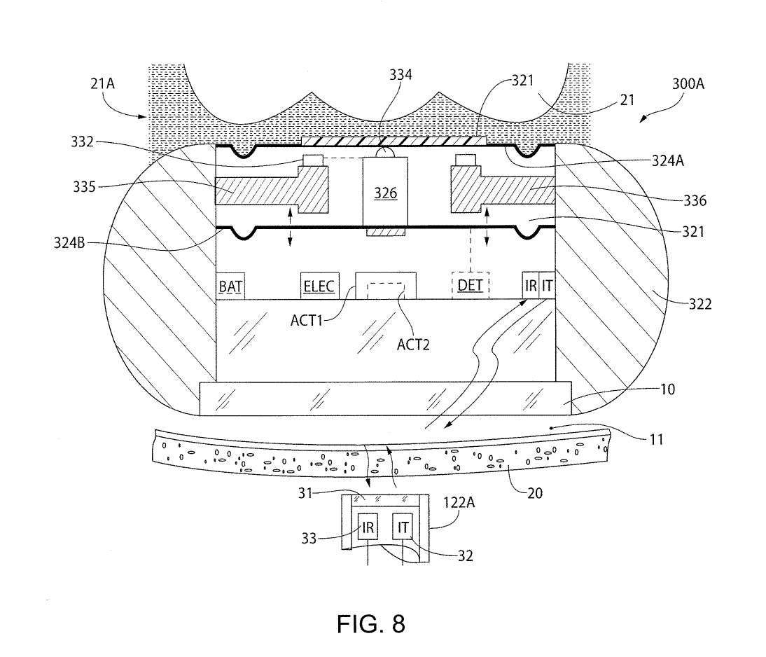

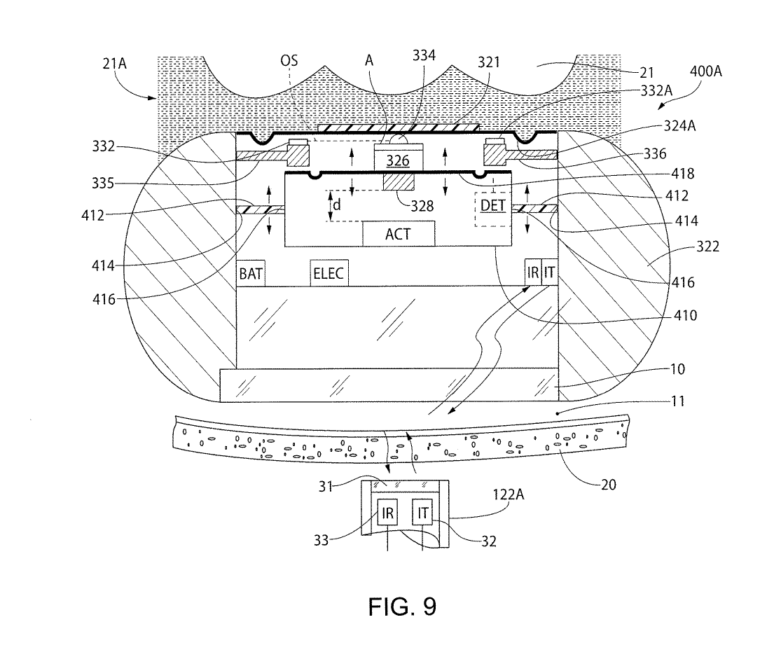

FIGS. 8-9 disclose a particular example of a communication interface CI in that the communication mechanism is an infrared communication mechanism. In particular, the sensor 300 or 400 may include a communication mechanism having an LED transmitter 8 (e.g., emitter OP200 by TT Electronics) and an LED receiver 9 (phototransistor OP500 by TT Electronics). Thus, measured internal pressure values can be detected by the sensor 300 or 400 and then transmitted out of the living being to a remotely-located infrared transceiver 122A. Similarly, the LED receiver 9 can be used to receive electromagnetic energy (e.g., infrared light) to charge the battery BAT or, if the implantable sensor is a passive device, to drive the electronics ELEC.

To effect the infrared communication, the side of the sensor housing 332 directly opposite the transmitter 8/receiver 9 pair comprises a transparent material (e.g., plexiglass) 10 that permits the passage of the infrared energy between the sensor 300/400 and the infrared receiver 122A. By way of example only, when an implantable sensor 300/400 is to measure intracranial pressure (ICP), the sensor 300/400 can be implanted within the subarachnoid space 11 of the test subject, outside the brain 21, the infrared energy passes through the scalp, skull, dura and arachnoid matter (the combination indicated by the reference number 20). The infrared receiver 122A also comprises an infrared transmitter 32/receiver 33 pair for communicating with an implantable sensor 300/400 and also includes a transparent distal end 31 for allowing passage of the infrared energy.

FIG. 10 provides a block diagram of the CI of the sensors 300/400 and the corresponding (optional) communication interface of the transceiver 122. The transducer's 326 electrical signal corresponding to the pressure is conditioned (e.g., amplified and filtered) by a signal conditioner and is digitized by the microcontroller 330 before being wirelessly transmitted (e.g., an ICP signal) to the transceiver 122A via the emitter LED 8. An LED receiver 33 then passes this to a microcontroller 131 for processing and ultimate display 133 or other output to the operator or user. An emitter LED 32 provides input/commands to the implantable pressure sensor 120A.

It should be noted, as mentioned earlier, that the microcontroller 330 controls the operation of the sensor 300/400, including the transducer electronics ELEC, the actuator ACT, and the emitter LED 8.

As mentioned earlier, pressure sensor 300/400 is powered from the internal battery BAT or from the receiver 122/122A utilizing electromagnetic waves (RF or IR) transmitted through the skin, tissue and/or bone. The measured quantity, e.g., pressure, is detected using an active sensor principle where the energy from the measured quantity is conditioned by the signal conditioner. In the preferred embodiment, information about the measured signal is converted to a frequency coded message and, for example, optically (e.g., infrared) transmitted outside the body to the receiver (see FIGS. 4A-4B, 7-15). When the transceiver 122A is activated by the user, the transceiver 122A sends an infrared pulse to the sensor 300/400. This signal wakes up (also referred to as a “start command”) the microcontroller 330 which controls the entire process in order to minimize power consumption. In particular, the steps to measure the signal by the sensor 300/400 are:
1) The microcontroller 330 turns on the pressure/force transducer 326 (e.g., piezoresistive die) and its signal conditioner;
2) Digitizing of the measured quantity (e.g., ICP) value;
3) Frequency modulating the measured (e.g., ICP) value;
4) Transmitting the frequency via infrared energy;
5) Implantable sensor goes to sleep.

One problem that this configuration encounters is the occasional occurrence of the output signal (i.e., the measured quantity signal 142) triggering the microcontroller 330 when the working wavelength of the “wake-up” signal 140 (e.g., transmitted infrared signal) and the measured quantity signal 142 (e.g., ICP signal) are the same. This problem is solved by two different methods. A first solution uses software whereby the microcontroller 330 overrides the wake up interruption signal 140 until the measured quantity signal 142 is sent; however this reduces the availability of ports in the microcontroller 330. A second solution is the use of two different wavelengths for signals 140 and 142 that do not interfere with one another. The latter solution is the preferred method since it takes advantage of some microcontroller inherent hardware benefits that prevents false triggering of the implantable sensor 300/400.

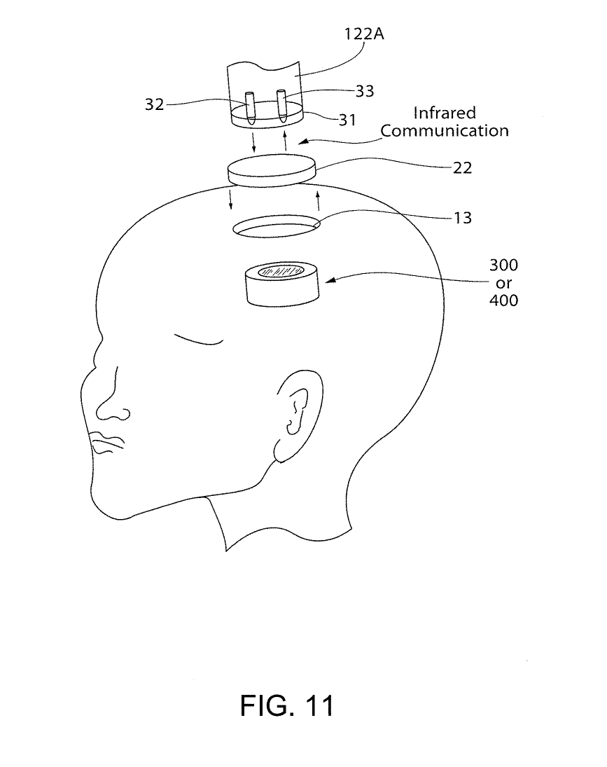

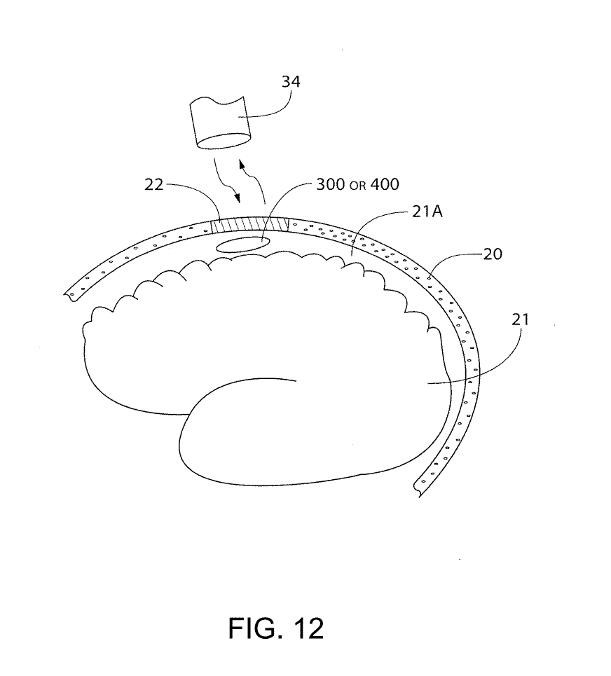

FIG. 11 depicts how an implantable sensor 300/400 can be positioned when used to measure ICP using implanted devices. In particular, a piece 22 of the skull is removed during trepanation to form a burr hole 13 and permit implantation of the sensor 300/400 in the brain, as discussed earlier with respect to FIG. 4-10. The sensor 300/400 is positioned with its transparent surface 10 facing outward to transmit/receive infrared energy outwardly of the skull towards the remotely-located transceiver 122A. Once the sensor 120A is positioned, the piece 22 of skull is re-inserted within the burr hole 13 and sensor 120A-transceiver 122A communication occurs as shown in FIG. 10. Therefore, although an implantable sensor 300/400 and the transceiver 122A require the use of respective transparent surfaces 10 and 31, infrared transmission through the scalp/skull/dura, arachnoid matter 20 does occur without major disruption of the infrared signals, as shown in FIG. 12.

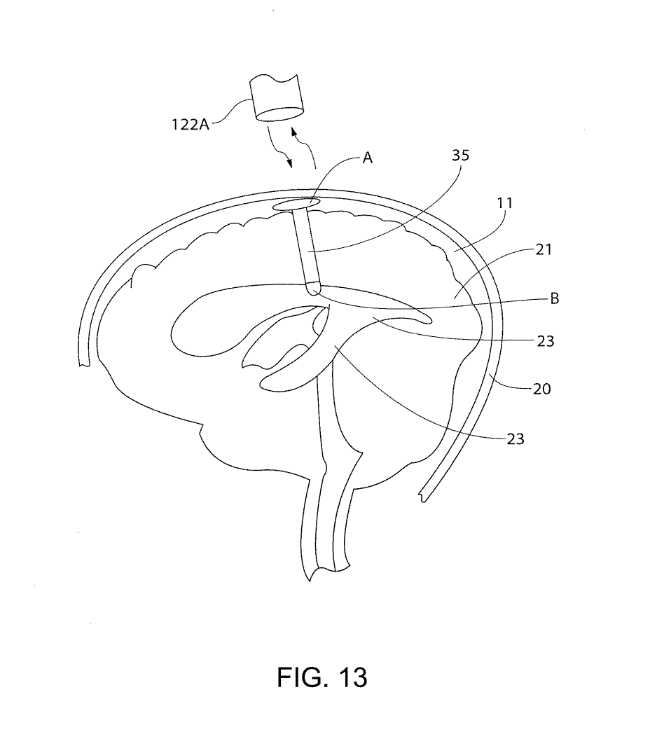

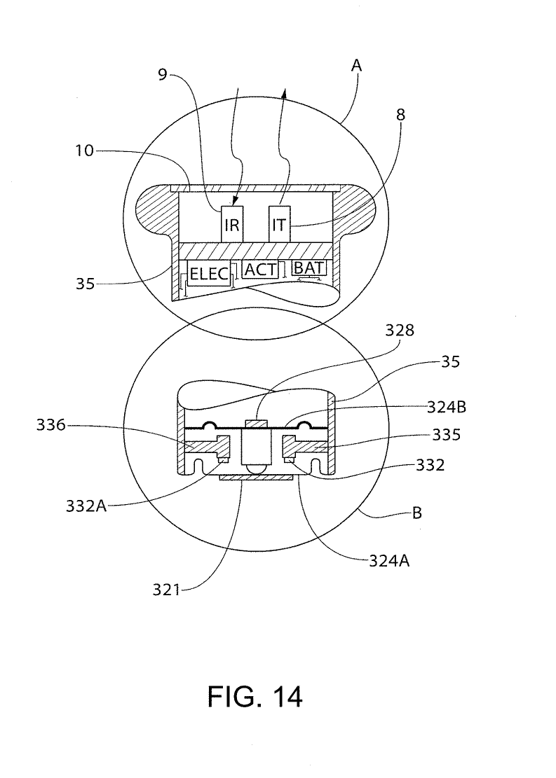

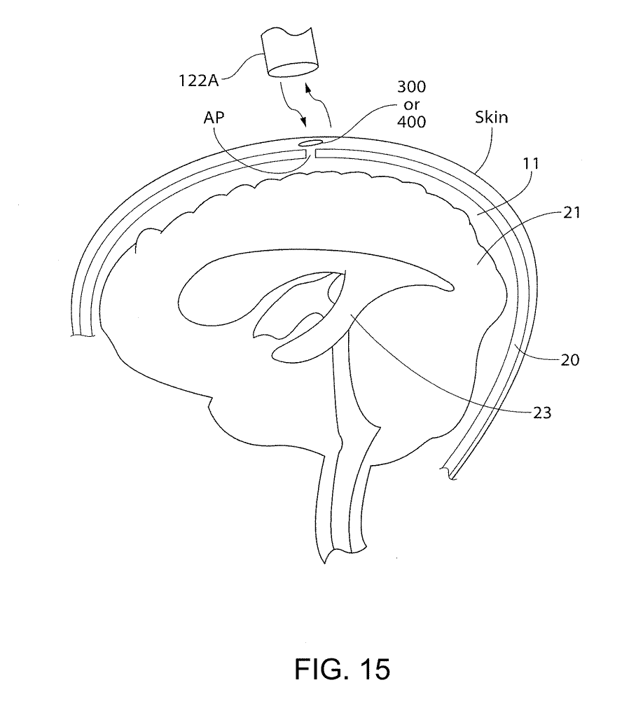

A further embodiment 120B, as shown in FIGS. 13-14, distributes an implantable sensor at the proximal and distal ends of a catheter 35. In particular, as shown most clearly in FIG. 13, the communication portion A of the sensor 300/400 is positioned at the proximal end of the catheter 35 which is located within the subarachnoid space 11; the pressure sensing portion B is located at the distal end of the catheter 35 within the brain ventricle 23 (FIG. 13). This configuration permits the pressure sensing portion B to be located within smaller and more critical areas of the brain without having to introduce the entire implantable pressure sensor 300/400 within such critical areas. It should be understood that the brain ventricle and subarachnoid space are shown by way of example only and that other implantation locations are within the broadest scope of the disclosure. In this embodiment, the communication portion A is located more closely to the outside of the living being to facilitate the wireless communication with the remotely-located transceiver 122/122A while permitting the pressure sensing to occur within a deeper location within the living being. An alternative example is shown in FIG. 15: an implantable sensor 300/400 may be positioned above the skull 20 but under the skin. A small aperture AP can be made in the skull to expose the implantable sensor 300/400 to the CSF and thus the ICP.

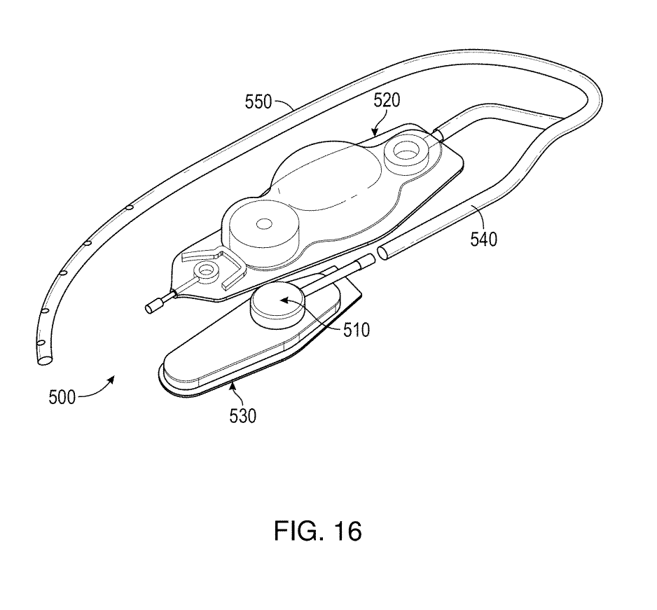

FIG. 16 shows a schematic view of a fluid draining device 500 comprising a shunt valve 520 for evacuating excess fluid (e.g., CSF or another body fluid) from an area of undesired excess fluid, such as a ventricle, a pressure sensor 530 mounted to a catheter 540 for monitoring pressure present the fluid (e.g., CSF or another body fluid), and a fluid drainage catheter 550. In this embodiment, the catheter may comprise a single lumen catheter, or it may comprise a dual lumen catheter having a dedicated catheter providing fluid contact (and pressure) to pressure sensor 530 and a discharge catheter for fluid drainage from the shunt valve 520. The pressure sensor chamber 510 of pressure sensor 530 is in communication with a lumen that fills with CSF (or another body fluid), and exposes a pressure-responsive membrane associated with the chamber directly to CSF (or another body fluid) under pressure. Pressure sensor 530 may comprise any of the pressure/force sensors described herein, and may be suitable for implementation of any of the calibration techniques described herein. In dual lumen catheter embodiments, a non-flowing lumen that communicates with the pressure sensor can be separated from the regular fluid stream so that the pressure measuring system stays patent. The pressure sensor can be interrogated non-invasively, for example, using an external handheld IR device.

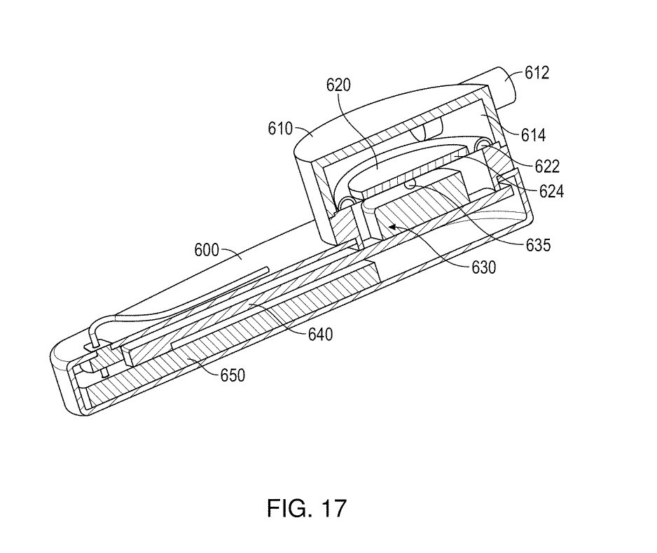

FIG. 17 shows a schematic cross-sectional view of another embodiment of a pressure sensor (e.g., an ICP or other body fluid sensor) as disclosed herein and suitable for use with fluid draining device 500. The pressure sensor illustrated comprises an external housing 600 having a generally cylindrical membrane/sensor/recalibration housing portion 610 and a port 612 for inflow and/or outflow of fluid (e.g., CSF or another fluid). An upper fluid chamber 614 of the membrane/sensor/recalibration housing 610 communicates with port 612 and is exposed to fluid (e.g., to CSF from the ventricle). A fluid-responsive sensor membrane 620 comprising a flexible peripheral rim 622 and a less flexible central portion 624 is mounted across the internal volume of membrane/sensor/recalibration housing 610, and a surface of sensor membrane 620 is exposed to upper fluid chamber 614. In some embodiments, the flexible peripheral rim of the sensor membrane comprises a thin titanium membrane (e.g., 50 microns thick). In some embodiments, central portion 624 of the sensor membrane is substantially stiff and may comprise a stiffening disk affixed to the flexible membrane. In some embodiments, the sensor membrane 620 is flat, e.g., has a planar configuration. In some embodiments, the sensor membrane 620 is relatively small, e.g., it has a diameter that is 6 mm or less; in some embodiments, the sensor membrane has a diameter of 5 mm or less; and in some embodiments, the sensor membrane has a diameter of 3 mm or less. In alternative embodiments, the sensor membrane for implantable sensors may have a diameter of at least 1 cm and up to 3 cm (e.g., from about 1 to 3 cm), and the sensor membrane for sensors used externally may have a diameter of at least 2.5 cm and up to 5 cm or more.

Force sensor 630 is mounted below fluid-responsive sensor membrane 620 and associated with an actuator 640 providing movement of sensor 630 toward and away from membrane 620. The sensor may comprise an HFD-500 Micro-Force Sensor (HDK America), for example, or a similar type of sensor. In one embodiment actuator 640 is a piezo-electric cantilever actuator mounted to a printed circuit board (PCB) 650 carrying the microprocessor, power system, and other system operating and energy components. The actuator may comprise a Piezodrive PL112 PICMA Bender Piezo Actuator (PI Ceramic GmbH) or a similar type of actuator. A known mass, illustrated in this embodiment as sphere 635, is mounted to or associated with force sensor 630 and is movable toward and away from sensor membrane 620. The mass of the known mass may vary depending on the system and its use as an implantable or external system; in general, objects having a mass of from about 0.05 g to 1.0 g are suitable. The actuator can reposition the sensor/mass away from the membrane for drift recalibration and sensor re-zeroing. The actuator can oscillate the sensor/mass at various amplitudes and frequencies, creating known forces, which can be used to recalibrate the device for drift (in the down position) and for biofouling (in the up position).

The microcontroller (e.g., a TI MSP430 ultra low power micro-controller or similar device) controls the calibration procedure, acquires analog signals from the sensor and communicates with a remote interface device. Power may be provided by a lithium ion battery that may be long-life or may be rechargeable using an IR laser in a remote device and IR LEDs in the sensor device for charging. The external housing, if the pressure sensor is implantable, is constructed from biocompatible materials and may use a two-layer structure comprising an internal hard capsule for electronics (e.g., epoxy resin) and a soft external shell (e.g., biocompatible polyurethane) for CSF channels, reservoirs, catheter connectors, etc. The external housing, if the pressure sensor is configured for external use, may be constructed from a variety of materials and may be vented to the atmosphere. The external housing, if the pressure sensor is configured for internal use, may be vented to surrounding medium such that the pressure inside the housing is the same as the pressure outside the housing. In alternative embodiments, the external housing may be hermetically sealed and, in embodiments in which the external housing is sealed, the housing may be filled with a liquid or a gas.

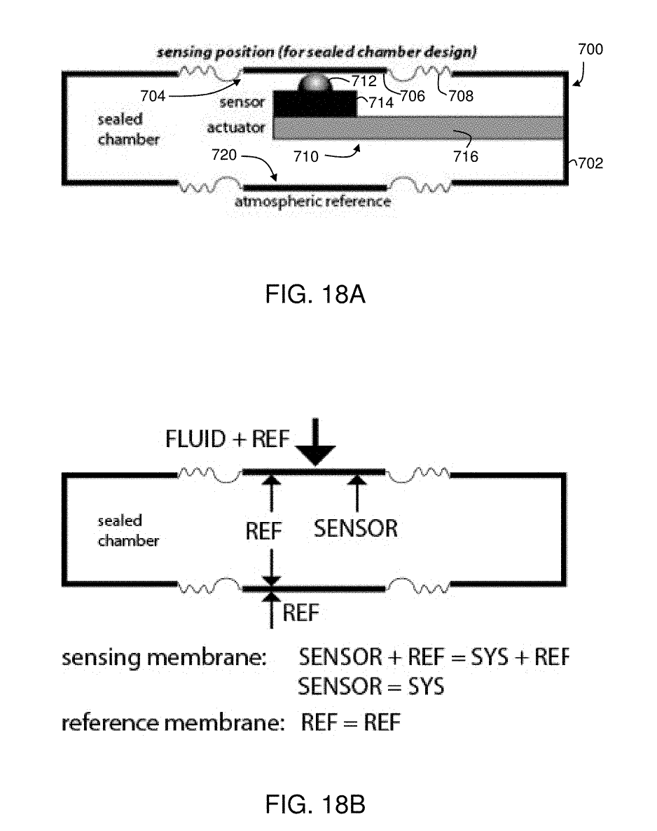

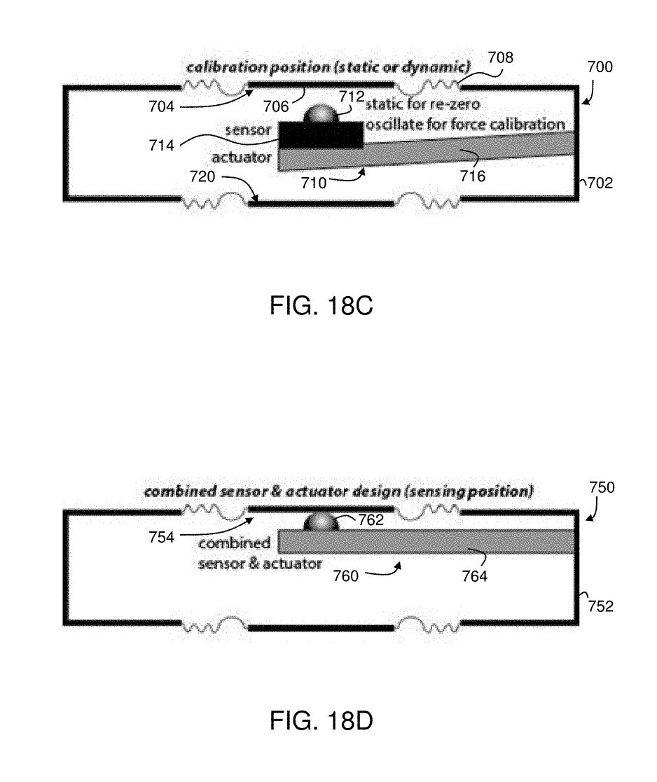

FIGS. 18A-D and 19A-B schematically illustrate additional pressure/force sensor embodiments that can be calibrated as described herein, and that are useful for measuring fluid pressure. These pressure-force sensors may be used in implantable pressure sensors for in situ pressure sensing of body fluids; in externally-positioned pressure sensing devices for contacting sensing body fluid pressures and/or for draining excess fluids; and in other types of pressure sensing devices. FIGS. 18A and 18C illustrate a pressure/force sensor 700 comprising an external sensor housing 702 having a first fluid-responsive membrane 704, and a movable actuator/sensor/mass combination 710 capable of being moved into and out of contact with the first movable membrane. An external surface of first fluid-responsive membrane 704 is configured to contact fluid whose pressure is desired to be detected, monitored or measured. In some embodiments, fluid-responsive membranes used in pressure-force sensors as described herein may comprise flexible and compliant diaphragm-type membranes. In some embodiments, fluid-responsive membranes used in pressure-force sensors may comprise substantially or partially rigid components (e.g., plates, diaphragms, etc.) mounted to the sensor housing via flexible or compliant interfaces. In the embodiments illustrated in FIGS. 18A-D and 19A-B, for example, first fluid-responsive membrane 704 comprises a plate 706 associated with housing 702 by means of a flexible or compliant peripheral member 708. It will be appreciated that plate 706 may be substantially rigid, it may be substantially planar or it may be curved or dome-like, and it may have a variety of configurations (e.g., circular). Compliant peripheral member 708 may comprise a compliant membrane (e.g., an annular membrane) having a variety of configurations, sizes and the like.

Oscillating actuator/sensor/mass combination 710 comprises an object 712 having a known mass, a force sensor 714, and an actuator 716 that is controllably movable (e.g., oscillatable) at predetermined or selectable frequencies and amplitudes. Actuator 716 is illustrated as a cantilevered lever, but it will be appreciated that other types of oscillating actuators may be used. For example, the actuator may comprise an electromagnet, or a microelectromechanical system (MEMS), piezoactuator, capacitive actuator, or other miniature actuator. In some embodiments, a sensor/mass combination may be mounted for movement (oscillation) on another (internal) movable membrane and moved by an actuator as described, above, in FIG. 4A.

As shown in FIGS. 18A-D, a fluid-responsive reference membrane 720 may be provided as a component associated with or integral to sensor housing 702, the fluid-responsive reference membrane 720 serving as a reference configured to be exposed to, and responsive to, a different fluid pressure condition, such as atmospheric pressure conditions, or pressure of a second fluid contacting the fluid-responsive reference membrane. In a sensor housing comprising a sensing membrane and a reference membrane, changes in surrounding pressure/pressure (e.g., atmospheric pressure, ATM) may be applied to both sensing surfaces, such that the force sensor only measures the pressure/force relative to the reference pressure/force. As shown in FIG. 18B, forces acting on the membranes include the reference pressure (REF), the system pressure which may include the pressure of the fluid (FLUID) and the reference pressure (REF), the opposing force provided by the force sensor (SENSOR), and the internal pressure of the housing. The presence of the reference membrane ensures that the pressure inside the housing is equal to the reference pressure (REF). In this case, the liquid or gas inside the housing transmits the pressure/force from the reference membrane to the inside surface of the sensing membrane, thus canceling the external reference pressure/force on the sensing membrane. Under normal sensing conditions, the sum of forces acting on the inside and outside of each membrane equals zero. Specifically, for the case of the sensing membrane, the internal pressure (REF) and the opposing force of the force sensor (SENSOR) act on the inside of the membrane and are equal to the system force (FLUID+REF) acting on the outside of the membrane; thus the sensor force SENSOR is equal to the fluid force FLUID. For example, in the case of typical ICP measurement the reference pressure is the atmospheric pressure ATM and the system pressure applied to the sensing membrane is the ICP plus atmospheric pressure ATM; thus, the force applied by/to the force sensor SENS is due only to the force from the ICP. Forces must be balanced as described, but it should be appreciated that forces can be calculated from pressures based on the area of the membranes (force equals pressure times area). The sensing membrane and the reference membrane can be the same size or different sizes and may have the same or different fluid-responsive characteristics, configurations and structures.

In the embodiment illustrated in FIGS. 18A-D, the movable actuator/sensor/mass combination is enclosed in a sealed chamber that may be filled with a liquid (e.g., silicone oil, mineral oil, alcohols such as glycerin, fluorocarbon-based fluids such as Fluorinert) or a gas (e.g., air, nitrogen, etc.). The actuator/sensor/mass combination is shown in FIG. 18A in a sensing position with the mass contacting a surface of the first fluid-responsive membrane. The actuator/sensor/mass combination is shown in FIG. 18C in a static or dynamic calibration position, in which the actuator/sensor/mass combination is out of contact with the fluid-responsive membrane. This position represents the static position for re-zeroing the sensor, and also represents one dynamic oscillation position used for force calibration.

FIG. 18D illustrates a similar pressure/force sensor embodiment, in which the sensor and actuator are combined in a single component and the mass is associated with the combined sensor and actuator. In this embodiment, pressure/force sensor 750 comprises an external sensor housing 752 having a first fluid-responsive membrane 754, and a movable actuator/sensor/mass combination 760 capable of being moved into and out of contact with the first fluid-responsive membrane. The oscillating actuator/sensor/mass combination 760 comprises an object 762 having a known mass, and a sensor/actuator combination 764, that is controllably movable (e.g., oscillatable) at predetermined or selectable frequencies and amplitudes. For example, a piezo bender can serve as both a sensor to measure force or deflection and as an actuator to provide movement (moving the sensor away from the membrane and/or applying an oscillation).

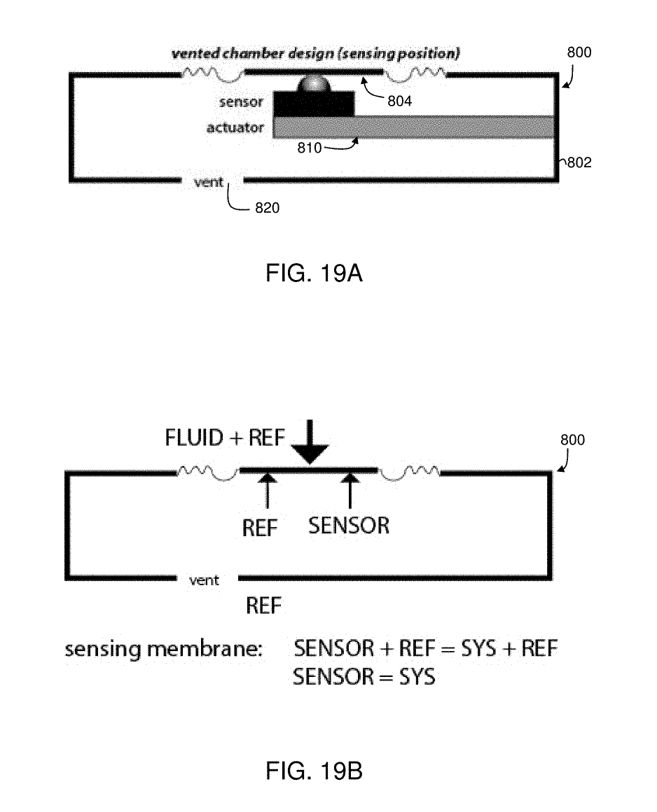

FIG. 19A illustrates another similar pressure/force sensor embodiment, with an actuator/sensor/mass combination in a sensing position, in which the sensor housing is vented. In this embodiment, sensor 800 comprises an external sensor housing 802 having a first fluid-responsive membrane 804, and a movable actuator/sensor/mass combination 810 capable of being moved into and out of contact with the first fluid-responsive membrane. An external surface of first fluid-responsive membrane 804 is configured to contact fluid whose pressure is desired to be detected, monitored or measured. The actuator/sensor/mass combination of FIG. 19A and the actuator/sensor/mass combination of FIG. 19A can be moved and vibrated as described for other housing arrangements to allow re-zeroing and calibration, including vibration of the sensor/mass with the sensor contacting the sensing membrane to calibrate for biofouling and vibration with the sensor/mass out of contact with the sensing membrane for force calibration.

Sensor 800 is vented to its external surroundings at vent 820, which may be an opening or a series of openings, or another structure that allows pressure communication between the housing interior and an external reference pressure REF (See, FIG. 19B). Under normal sensing conditions, the sum of forces acting on the inside and outside of the sensing membrane is zero. Specifically, the internal pressure (REF) and the opposing force of the force sensor (SENSOR) act on the inside of the membrane and are equal to the system force (FLUID+REF) acting on the outside of the membrane; thus the sensor force SENSOR is equal to the fluid force FLUID. For example, in the case of typical ICP measurement the reference pressure is the atmospheric pressure ATM and the system pressure applied to the sensing membrane is the ICP plus atmospheric pressure ATM; thus, the force applied by/to the force sensor SENS is due only to the force from the ICP.

The vented chamber designs of FIGS. 19A, B can also be applied to the devices described and illustrated above, e.g., FIG. 4. For example, a vent or a reference membrane can be added to the housing 322 to provide pressure communication between the chamber 321 and the external reference pressure REF. In either case, the membrane 324B should allow for pressure communication between the chambers on either side of membrane 324B, for example by providing holes in the membrane 324B or a fluid passage connecting the two chambers.

In the description provided above, the term “about” means +/−20% of the indicated value or range unless otherwise indicated. The terms “a” and “an,” as used herein, refer to one or more of the enumerated components or items. The use of alternative language (e.g., “or”) will be understood to mean either one, both, or any combination of the alternatives, unless otherwise expressly indicated. The terms “include” and “comprise” and “have” are used interchangeably and both of those terms, and variants thereof, are intended to be construed as being non-limiting. The term “contact” and derivative terms are used to include both direct contact and indirect contact with an object or surface. The terms “flexible membrane” and “fluid-responsive membrane” are used interchangeably to refer to a surface that moves, and/or flexes, in response to changes in pressure exerted on the membrane by a fluid (e.g., liquid or gas).

It will be appreciated that the methods and systems of the present invention may be embodied in a variety of different forms, and that the specific embodiments shown in the figures and described herein are presented with the understanding that the present disclosure is considered exemplary of the principles of the invention, and is not intended to limit any claimed subject matter to the illustrations and description provided herein. The various embodiments described may be combined to provide further embodiments. The described devices, systems, methods and compositions may omit some elements or steps, add other elements or steps, or combine the elements or execute steps in a different combination or order than that specifically described.

We claim:

1. A method comprising: disposing a pressure sensor comprising a first fluid-responsive membrane forming a portion of the pressure sensor in contact with a fluid whose pressure is desired to be detected; displacing a force transducer mounted in a housing of the pressure sensor from a position in contact with the first fluid-responsive membrane to a position out of contact with the first fluid-responsive membrane; and re-calibrating the pressure sensor to a zero point corresponding to the force transducer measurement at the position out of contact with the first fluid-responsive membrane.

2. A method for calibrating a pressure sensor comprising: oscillating a force transducer at a first amplitude and a first frequency, wherein the force transducer is located within a pressure sensor internal chamber, and wherein the pressure sensor comprises a housing having a first fluid-responsive membrane configured to contact a fluid of interest; collecting a force data set corresponding to force measured during oscillation of the force transducer; calculating the theoretical force exerted on the force transducer as a result of oscillation and comparing the theoretical force to the force data set; and deriving a calibration characteristic based on the comparison.

3. The method of claim 2, comprising collecting a plurality of force data sets corresponding to force measured during oscillation of the force transducer at a plurality of different amplitudes and/or frequencies and deriving the calibration characteristic based on a plurality of dynamic force data sets.

4. The method of claim 2, wherein the pressure sensor internal chamber is sealed.

5. The method of claim 2, wherein the pressure sensor internal chamber is vented.

6. The method of claim 2, comprising oscillating the force transducer at a first amplitude and a first frequency and collecting the force data set while the force transducer contacts the first fluid-responsive membrane.

7. The method of claim 2, comprising oscillating the force transducer at a first amplitude and a first frequency and collecting the force data set while the force transducer is out of contact with the first fluid-responsive membrane.

8. The method of claim 2, comprising oscillating the force transducer and generating a calibration characteristic to recalibrate for drift and re-zero the sensor in one position of the force transducer and oscillating the force transducer and generating a calibration characteristic to recalibrate the sensor for biofouling in another position.

9. A pressure sensor comprising a housing, a fluid-responsive sensor membrane associated with the housing and configured for contacting a fluid whose pressure is desired to be detected, a force transducer positioned within the housing and configured for measuring force exerted on the transducer, and an actuator associated with the force transducer and configured to oscillate the force transducer within the housing.

10. The pressure sensor of claim 9, wherein the housing is sealed, forming an internal cavity in with the force transducer is positioned, and the internal cavity is fluid-filled.

11. The pressure sensor of claim 10, wherein the internal cavity is filled with one of a liquid and a gas.

12. The pressure sensor of claim 9, additionally comprising a reference fluid-responsive sensor membrane associated with the housing and configured for contacting a reference fluid external of the housing.

13. The pressure sensor of claim 9, wherein the housing has a vent providing communication between an internal cavity of the housing and an external environment.

14. The pressure sensor of claim 9, additionally comprising an object having a known mass associated with the force transducer.

15. The pressure sensor of claim 9, wherein the actuator and the force transducer are provided as a single component.

16. The pressure sensor of claim 9, wherein the fluid-responsive membrane is in communication with a fluid lumen configured to contain the fluid whose pressure is desired to be detected.

17. The pressure sensor of claim 9, wherein the fluid-responsive sensor membrane comprises a flexible peripheral rim mounted to the housing and a less flexible central portion associated with the flexible peripheral rim.
